(19) United States
(12) Patent Application Publication
(10) Pub. No.: US 2025/0295215 A1
(43) Pub. Date: Sep. 25, 2025

(54) Manual Applicator For Cosmetic Product

(71) Applicant: ELC MANAGEMENT LLC., Melville, NY (US)

(72) Inventors: Matthew Morris Chateauvert, Port Chester, NY (US); David Edward Wilson, Plainview, NY (US); Wilson A. Lee, Hauppauge, NY (US)

(21) Appl. No.: 18/614,064

(22) Filed: Mar. 22, 2024

(51) Int. Cl. A45D 34/04

(52) U.S. Cl. CPC A45D 34/042; A45D 2200/25

(57) ABSTRACT

A system for applying a cosmetic substance may include a handheld device with an elongated portion capable of being grasped by a user, a main head body portion coupled to the elongated portion, and an applicator tip seated on the top side of the main head body. The applicator tip is capable of supporting the cosmetic substance to be applied to the skin. A kit may include the system for applying a cosmetic substance and one or more chips comprising the cosmetic substance to be applied to the skin. The one or more chips may include at least one microneedle composed of the cosmetic substance to be applied to the skin. The handheld device of the system may be capable of inserting the at least one microneedle into an epidermal layer of the skin. Each chip may include an array of microneedles composed of the cosmetic substance.

### FIELD OF THE DISCLOSURE

The present disclosure generally relates to cosmetic, hair care, body care, and/or skincare products and, more particularly, to systems, devices, and approaches for applying such products.

### BACKGROUND

Cosmetic and/or skincare products may be provided in a number of forms and may be applied using a number of varying approaches. For example, toner or watery lotion may be applied by spraying or dabbing on with fingers or a cloth pad and applying to skin. Creams may be dispensed from a container and applied with fingers or brushes topically to skin. These conventional methods of storing and dispensing cosmetics and skin care products typically apply cosmetic substances to the uppermost layer of the skin. The active ingredients may reach the deeper layers of the skin in doses which are lower than in the topically applied cosmetic substance. In the case of hygroscopic or moisture sensitive products storage and the form of the cosmetic substance may affect the efficacy of the active ingredient when the cosmetic substance is applied to a user's skin. For example, granules or anhydrous micro-structures stored with desiccants may ensure the stability of a product longer than a cream stored in a tub or a liquid in a bottle that is exposed to ambient air. Further, the conventional methods applying cosmetic substances can lack precise control over the area affected by the active ingredients, potentially also leading to wasted product.

Accordingly, there is a need for systems with improved functionalities for applying cosmetic substances.

### SUMMARY

Examples within the scope of the present disclosure are directed to a handheld device for applying a cosmetic substance to skin. The handheld device includes an elongated portion capable of being grasped by a user, a main head body, and an applicator tip. The main head body includes a biasing mechanism and is operably coupled with the elongated portion. The main head body has a top side to which the applicator tip is capable of being sit upon. The applicator tip is also capable of supporting a cosmetic substance to be applied to the skin.

In an approach, the biasing mechanism of the main head body is capable of restricting infiltration of the cosmetic substance into an epidermal layer of the skin until a predetermined threshold force is applied to the applicator tip. The predetermined threshold force may be a predetermined minimum threshold force which must be overcome in order for the cosmetic substance to be applied to the epidermal layer. Alternatively, the predetermined threshold force may be a maximum threshold force above which the cosmetic substance is prevented from further infiltrating the skin. In some such approaches, the handheld device is operable to deliver the cosmetic substance to a depth of between approximately 10 μm (micrometers) and approximately 45 μm into the epidermal layer.

In these and other approaches the handheld device further includes a feedback indicator that is capable of providing an indication when a predetermined maximum force is applied to the applicator tip. In some such approaches, the indication includes at least one of a sound, a clicking sensation, an audible click, or a resistive force. The resistive force may be caused by a detent in the handheld device between the main head body and the elongated portion. In some such approaches, the predetermined maximum force is a force beyond which damage to the skin and tissues beneath the skin may occur.

Further, in these and other approaches the cosmetic substance has a microstructure, such as a microcrystalline structure. Additionally, or alternatively, the handheld device is operable to deliver the cosmetic substance through depressing the applicator tip against the skin with a first pressure greater than a threshold minimum pressure until a second pressure less than or equal to a predetermined maximum force is applied to the skin, in which the handheld device is capable of being held by a user applying pressure.

In some approaches, the handheld device has a raised portion of the main head body, and the raised portion encircles at least a portion of the applicator tip. Further, in some such approaches, the raised portion of the main head body has a free end which extends from the main head body beyond the applicator tip when no force is applied. The main head body head tapers from a first outer diameter to a second outer diameter at the free end. The raised portion of the main head body is capable of pulling the skin taut when force is applied to the skin during delivery of the cosmetic substance.

An example method for delivering a cosmetic substance to skin includes attaching a chip with the cosmetic substance to an applicator tip of a handheld device, contacting a main head body of a handheld device to the skin, moving an elongated portion of the handheld device toward the skin in response to haptic feedback, reversing motion of the elongated portion such that the handheld device is moving away from the skin, and repetition of the motion of the elongated portion toward the skin and away from the skin.

In an approach, the method includes delivery of the cosmetic substance in a single location of the skin. In this and other approaches, the repetition of the motion of the elongated portion of the handheld device is determined by a user of the handheld device.

A kit for applying a cosmetic substance to skin includes a handheld device for applying the cosmetic substance and a chip comprising the cosmetic substance to be applied to the skin in some examples.

In an approach, in the kit, the handheld device may include a portion capable of being grasped by a user, a main head body, and an applicator tip. The portion capable of being grasped by a user is an elongated portion in some approaches. In these and other approaches, the main head body includes a biasing mechanism and is operably coupled with the portion capable of being grasped by a user. The applicator tip is capable of being seated on a top side of the main head body and is also capable of supporting the cosmetic substance to be applied to the skin.

In another approach, the chip includes a microneedle composed of the cosmetic substance. In these and other approaches, the handheld device is capable of inserting the microneedle into an epidermal layer of the skin when a pressure at or above a predetermine threshold minimum pressure is exerted on the applicator tip of the handheld device. The handheld device is capable of inserting the microneedle to a depth of between approximately 30 μm (micrometers) and approximately 50 μm into the epidermal layer in some approaches. The chip is part of an array of chips that include the cosmetic substance in various approaches.

### BRIEF DESCRIPTION OF THE DRAWINGS

The above needs are at least partially met through provision of one, more than one, or any combination of the approaches for systems for containing and dispensing a cosmetic product described in the following detailed description, particularly when studied in conjunction with the drawings, wherein:

FIG. 1A illustrates a side view of an example system including a hand-held device for applying a cosmetic substance in accordance with various examples;

FIG. 1B illustrates a side elevation view of the example system of FIG. 1A with a main head body in a compressed configuration in accordance with various examples;

FIG. 2 illustrates a side perspective view of the example system of FIGS. 1A and 1B in accordance with various examples;

FIG. 3 is an exploded view of the example system of FIG. 1A in accordance with various examples;

FIG. 4 illustrates a perspective view of a portion of the example system of FIGS. 1A-3 with a main head body portion removed from the remaining portion of the system in accordance with various examples;

FIG. 5 illustrates a perspective view of a main head body portion of the example system of FIGS. 1A-3 in accordance with various examples;

FIG. 6A illustrates a perspective view of an applicator tip of the example system of FIGS. 1A-3 in accordance with various examples;

FIG. 6B illustrates a side elevation view of an applicator tip of the example system of FIGS. 1A-3 in accordance with various examples;

FIG. 7A illustrates an applicator tip with an array of microstructures comprising a cosmetic substance to be applied to skin in accordance with various examples;

FIG. 7B is an enlarged view of the applicator tip with cosmetic substance microstructure array seen in FIG. 7A;

FIG. 8 illustrates a cross-sectional view of the example system of FIG. 2, taken along the line A-A, in accordance with various examples; and

FIG. 9 illustrates a cross-sectional view of an example system with a spring biasing mechanism in accordance with various examples;

FIG. 10 illustrates a cross-sectional view of an example system with a spring biasing mechanism and a sensory feedback mechanism in accordance with various examples;

FIG. 11 illustrates a cross-sectional view of an example system with a magnet-based biasing mechanism in accordance with various examples;

FIG. 12 illustrates a cross-sectional view of an example system with a ratcheting mechanism in accordance with various examples;

FIG. 13 illustrates an array of microstructures, microneedles, comprising a cosmetic substance applied to an epidermal layer of a user's skin using an example system in accordance with various examples;

FIG. 14 illustrates a side view of an example system for applying a cosmetic substance in accordance with various examples;

FIG. 15 illustrates a top perspective view of the example system of FIG. 14 in accordance with various examples;

FIG. 16 is an exploded view of the example system of FIGS. 14 and 15 in accordance with various examples;

FIG. 17 illustrates a side elevation view of the example system in accordance with various examples;

FIG. 18 illustrates a cross-sectional view of the example system of FIG. 17, taken along the line B-B, in accordance with various examples;

FIG. 19 illustrates a side elevation view of the main head body of the example system of FIG. 16 in accordance with various examples;

FIG. 20 is a cross-sectional view of the main head body shown in FIG. 19 taken along the line C-C, in accordance with various examples;

FIG. 21 illustrates an example array of microstructures in the form of microneedles, comprising a cosmetic substance applied to an epidermal layer of a user's skin using the example system of FIG. 14, in accordance with various examples;

FIG. 22 illustrates a side elevation view of an example system for applying a cosmetic substance in accordance with various examples;

FIG. 23 illustrates a cross-sectional view of the system of FIG. 22 in accordance with various examples; and

FIG. 24 illustrates an example method for using the systems and apparatus for applying a cosmetic substance in accordance with various examples.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible examples are often not depicted in order to facilitate a less obstructed view of these various examples. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various approaches, systems, apparatuses, kits, and methods for applying a cosmetic substance are provided that allow the cosmetic substance to be administered to an epidermal layer of a user's skin. In some examples, the system may include a handheld device that includes a portion that is grasped by a user, and a removable component, such as an applicator tip, that allows for utilization of different cosmetic substances. The ability of the system to be used with disposable chips containing cosmetic substances can also enable the system to be used with diverse types of cosmetic substances.

Turning to the Figures, a system 100 is provided for applying a cosmetic substance to an epidermal layer of the skin. The system 100 includes a handheld device with an elongated portion 110, a main head body 120, and an applicator tip 130. The elongated portion 110 is capable of being grasped by a user when applying a cosmetic substance 105 that is retained or otherwise disposed on a first (upper) surface 131 of the applicator tip 130 to the user's skin. The main head body 120 has a first surface (top side) 121 on which the applicator tip 130 is situated when the system 100 is in use.

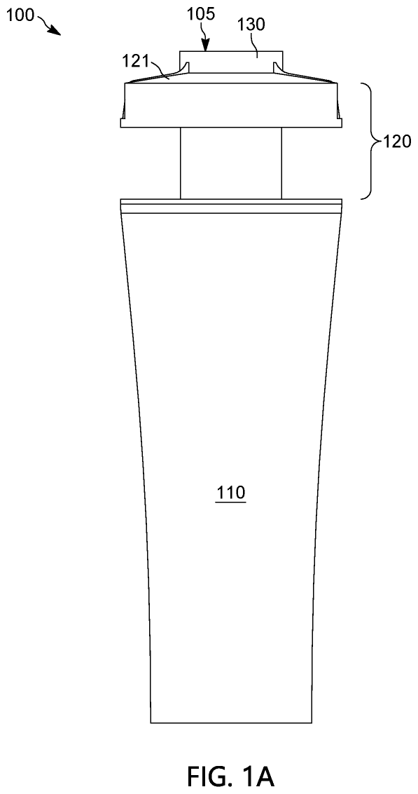

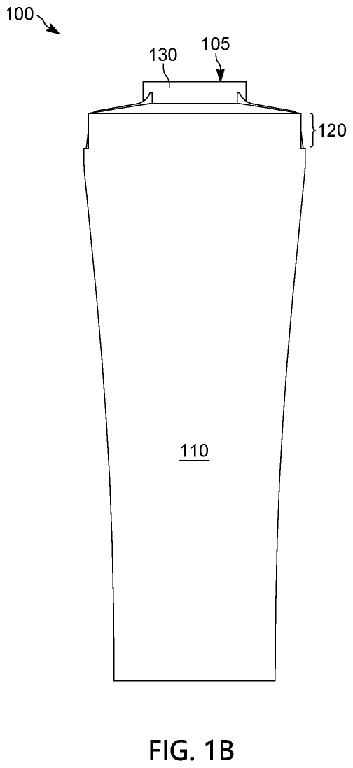

As can be seen in FIGS. 1A and 1B, the system can be arranged in a compressed configuration (FIG. 1B) or in an extended configuration (FIG. 1A). The main head body 120 includes a biasing mechanism which enables the transition of the system from the compressed configuration to the extended configuration.

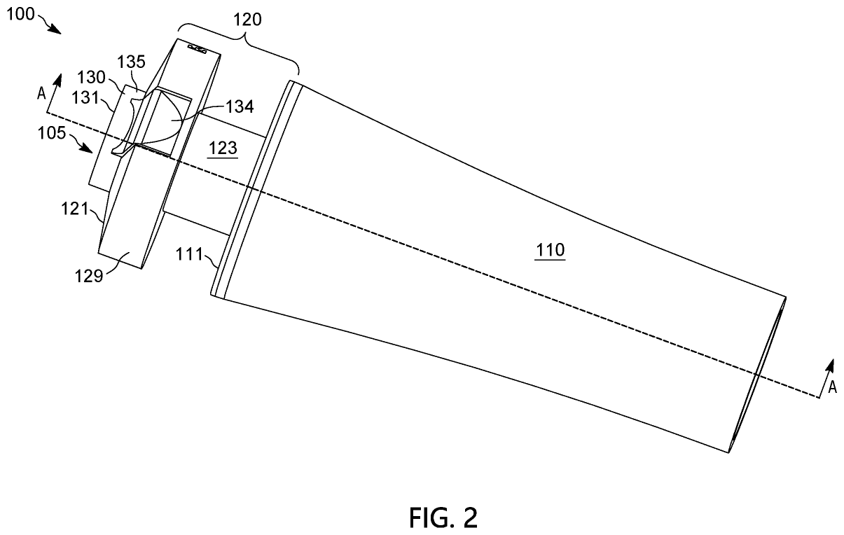

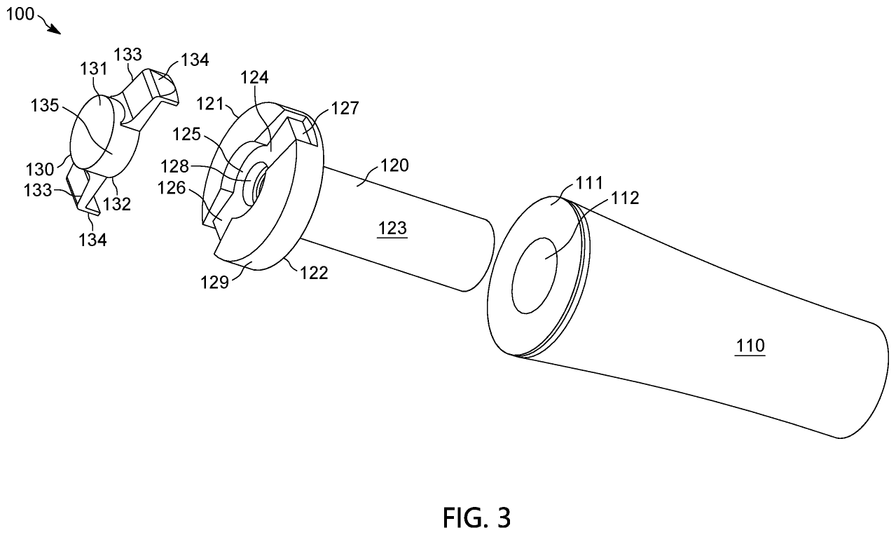

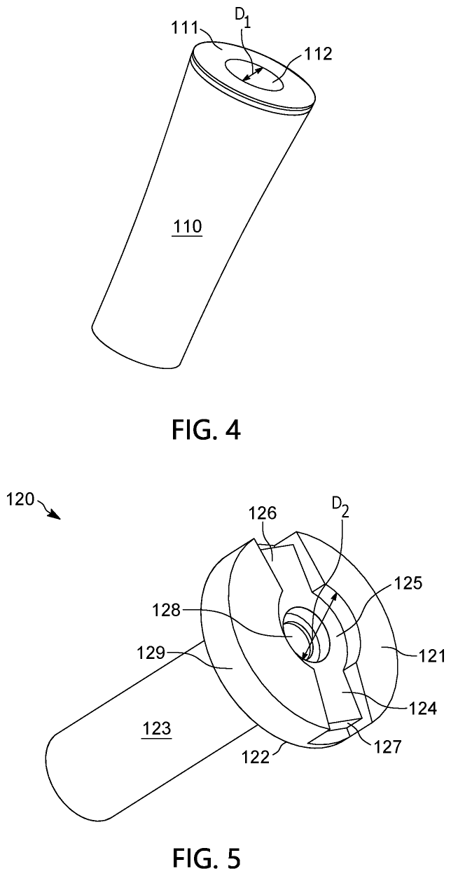

FIG. 2 is a side view of the elongated portion 110, main head body 120, and applicator tip 130 assembled and in the extended configuration, and FIG. 3 is an exploded view of the system 100 that includes these components. The elongated portion 110 receives a stem 123 of the main head body 120 in a central aperture 112 when the system 100 is assembled. The stem 123 of the main head body 120 has an outer diameter which corresponds to a diameter D₁ (shown in FIG. 4) of the central aperture 112 of the elongated portion 110 of the handheld device. In the compressed configuration, the first surface 111 of the elongated portion abuts and/or is positioned near a second surface (lower side) 122 of the main head body 120.

The main head body 120 has a recess 124 for receiving the applicator tip 130. The recess 124 allows the applicator tip 130 to be inserted from the first surface 121 of the main head body 120. The shape of the recess 124 is contoured to accept the applicator tip 130. As seen in FIGS. 2, 3, and 5, the recess 124 in the main head body 120 has a central portion 125, side portions 126, and tab receiving portions 127. The central portion 125 is flanked by the side portions 126, one on either side. It is to be appreciated that in some examples, the recess 124 may have any number of alternative shapes or arrangements for receiving various alternative shapes, arrangements, or different configurations of the applicator tips.

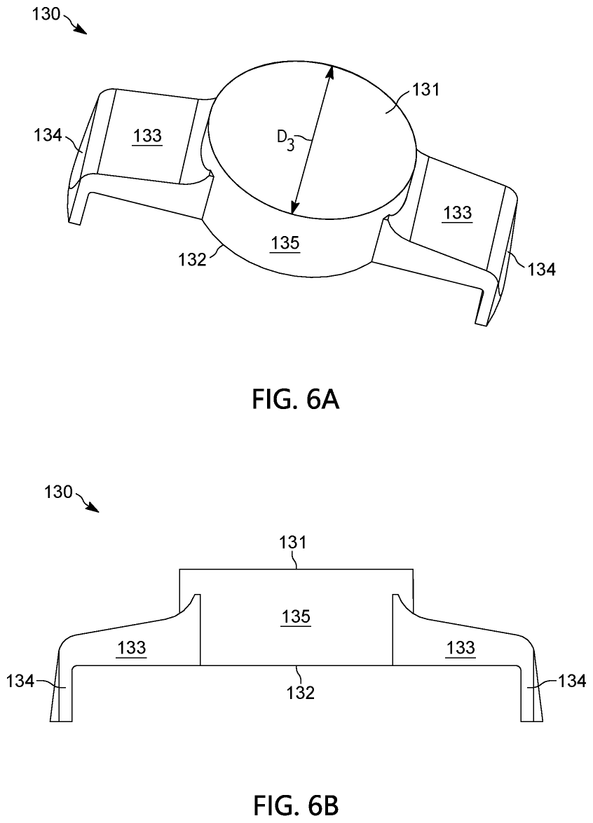

In FIGS. 2, 3, and 5, the side portions 126 of the recess 124 are situated linearly, 180 degrees apart from each other about the central portion 125. The recess 124 and the relative locations of the central portion 125 and side portion 126 may differ from that shown in the figures to accommodate the configuration of the applicator tip 130. The central portion 125 has a diameter D₂ which is approximately the same as the diameter D₃ (shown in FIG. 6A) of a central portion 135 of the applicator tip 130. Each side portion 126 has the central portion 125 at one end and a tab receiving portion 127 at the other end. Each tab receiving portion 127 extends from the first surface 121 of the main head body toward the second surface 122 in a sidewall 129 of the main head body 120. In the center of the main head body 120 is a channel 128 that extends from the recess 124 through all or a portion of the length of the stem 123.

The main head body 120 supports an applicator tip 130 when the system 100 is assembled for use. The applicator tip 130 is shown in greater detail in FIGS. 6A and 6B. The applicator tip 130 has the previously-noted first surface (top side) 131, a second surface (bottom side) 132, a central portion 135, side portions 133, and tabs 134. On the applicator tip 130, the central portion 135 is flanked by two side portions 133, one on each side so that the side portions 133 and the central portion 135 are aligned linearly. At the outer extremity of each side portion 133 is a tab 134. Each tab 134 extends in a downward fashion. The second surface 132 of the applicator tip 130 is in contact with the first surface 121 of the main head body 120 when the system 100 is assembled. Like the recess 124, it is to be appreciated that in some examples, the applicator tip 130 may have any number of alternative shapes or arrangements which accommodate different configurations of the recess 124.

As described above, the recess 124 of the main head body 120 is shaped and dimensioned to receive the applicator tip 130 when the system is assembled for use. The central portion 135 of the applicator tip 130 is seated in the central portion 125 of the recess 124 in the main head body 120, and, correspondingly, the side portions of the components align, and the tabs 134 of the applicator tip slot into the tab receiving portions 127. To that end, the diameter D₃ of the applicator tip central portion 135 is less than or equal to the diameter D₂ of the central portion 125 of the main head body 120.

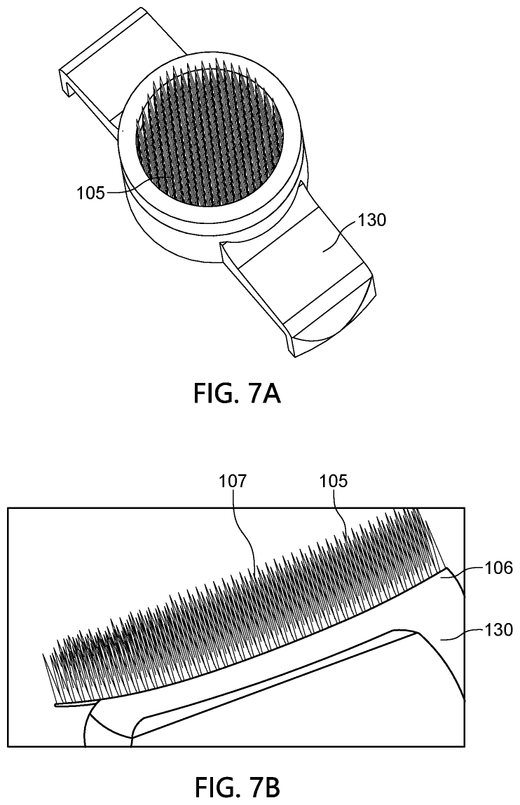

When assembled for use, the first surface 131 of the applicator tip will have a cosmetic substance 105 disposed thereupon. An applicator tip 130 with cosmetic substance 105 on the first surface 131 is shown in FIGS. 7A and 7B. The cosmetic substance 105 is in the form of an array of microstructures 107 supported by a substrate or chip 106. The array of microstructures may be an array of microneedles. The microneedles may be solid or hollow. In some examples, the microstructures are hollow microneedles which allow the cosmetic substance 105 to be delivered to a desired depth into the user's skin by flowing the cosmetic substance through the microneedles. Alternatively, in some examples, the microstructures are solid microneedles that are coated with the cosmetic substance 105. In other examples, the microstructures are dissolvable microneedles composed of the cosmetic substance. In some examples, the chip of substrate 106 may be a solid form of the cosmetic substance, while in some other examples the chip or substrate 106 may be another material such as a polymer, ceramic, metal foil, semiconductor, or another material which is inert with respect to the cosmetic substance and on which microstructures may be formed.

In examples where the cosmetic substance is present as an array of dissolvable microneedles, the cosmetic substance 105 may be in a glassy or microcrystalline state, where the cosmetic substance is solid at ambient temperatures (e.g., up to approximately 40 degrees C., preferably under 40 degrees C.) and ambient relative humidity, but once inserted into the epidermis of the skin, the inserted portion of the cosmetic substance microneedle will dissolve, releasing the active ingredient of the microneedle. Alternatively, the microneedles may be composed of the cosmetic substance which may be sensitive to moisture in the air (e.g., humidity), and due to this sensitivity, the chip 106 including the array of microstructures 107 may be preserved in a dry environment where the humidity is near zero. Maintaining a dry environment may involve controlling the temperature of the storage area of the cosmetic substance. For example, in a colder environment, the air is not capable of retaining as much moisture as in a warmer environment, and thus the colder the environment the less moisture is in the air, or surrounding atmosphere, and is available to affect the cosmetic substance. Desiccant, vacuum packaging, inert gas, or any combination of those may be used with or without temperature control to maintain a dry environment for the chip 106 and cosmetic substance 105.

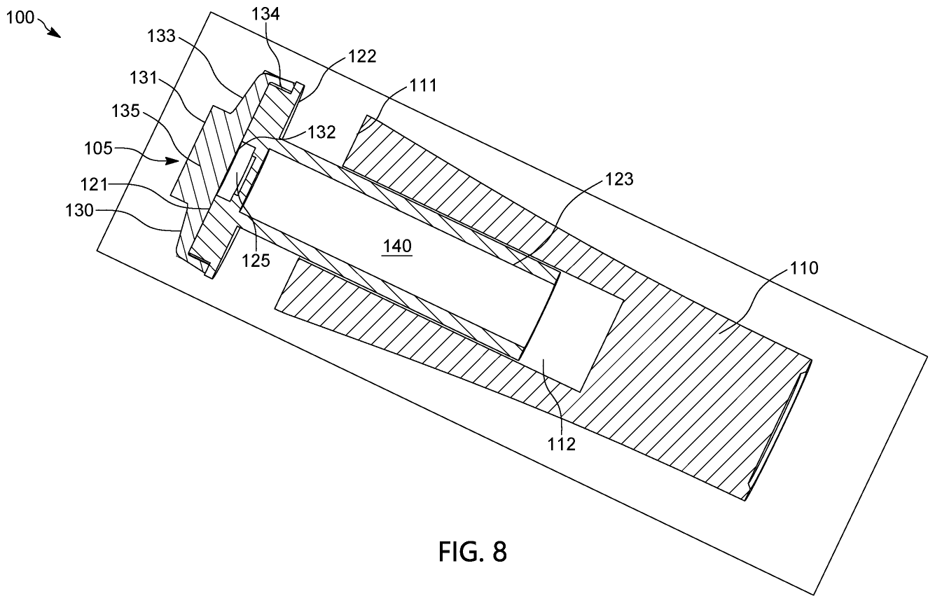

FIG. 8 is a cross-sectional view of the assembled system 100 of FIG. 2 taken along the line A-A. In FIG. 8, assembled system 100 is in an extended configuration, with the elongated portion 110 having its first surface 111 separated from the second surface 122 of the main head body 120. The stem 123 of the main head body 120 is inserted into the elongated portion 110 in the central aperture 112. The stem 123 of the main head body 120 is shown as hollow, such that a volume 140 in the system 100 is present that is bounded partially by the second surface 122 and the channel 128 of the main head body 120. The volume 140, the exterior of the stem 123, and the interior of the central aperture 112 are areas which are utilized in the system as locations for any combination of biasing mechanisms, biasing components, feedback mechanisms, and feedback components; in some example systems, these areas a locations for mechanisms and/or components which provide both biasing and sensory feedback to a user.

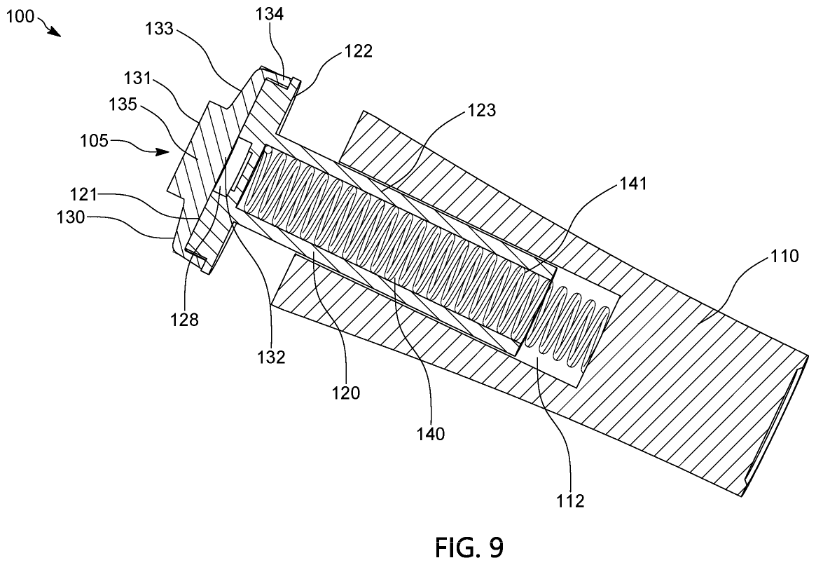

FIGS. 9-12 are cross-sectional views of various example systems 100 with biasing and feedback mechanisms or components. Biasing mechanisms or components may urge the main head body 120 away from the elongated portion (e.g., handle) 110. Alternatively, biasing mechanisms or components in the various systems can be considered to provide resistance or force between the main head body 120 and the elongated portion 110 when the first surface 131 of the applicator tip 130 is pressed against skin. In examples, feedback mechanisms or components provide sensory (e.g., haptic) feedback to a user, giving the user an indication of when sufficient force has been applied in the use of the system to urge the cosmetic substance 105 in the form of an array of microstructures 107 through the top portion of a typical epidermal layer. In some example systems, the feedback indicator mechanisms or components may be operable to provide an indication when a predetermined maximum force is applied to the applicator tip 130. The predetermined maximum force may be a force beyond which damage to the skin and tissues beneath the skin may occur. Alternatively, or additionally, in some example systems, the biasing mechanisms or components of the systems described herein may be capable of inserting a microneedle of a microstructure array into an epidermal layer of the skin when a pressure at or above a predetermined threshold minimum pressure is exerted on the applicator tip.

A spring 141 is the biasing mechanism shown in FIG. 9. The spring 141 is situated within the volume 140 created by the main head body stem 123 and the central aperture 112 in the elongated portion. The spring 141 is selected for its resilience, weight, and the ability to apply an appropriate amount of force to the chip 106 carrying the cosmetic substance 105 for appropriate penetration of the microstructures 107 into the skin. It may be desirable to have a spring that is lighter in weight so that the over-all weight of the system does not exceed a predetermined threshold value, such as approximately 0.5 kilograms or approximately 0.3 kg.

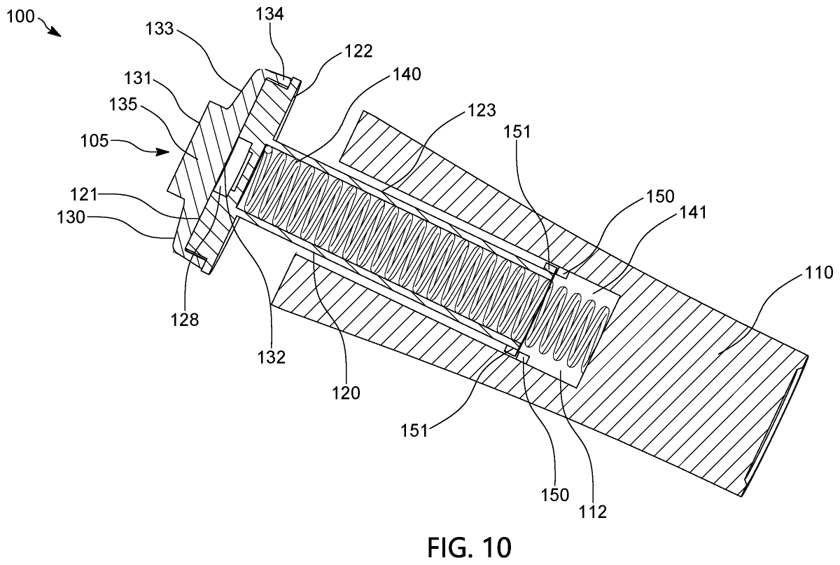

FIG. 10 shows an example system with a spring 141 as a biasing component with feedback components 150, 151. The feedback components 150, 151 may include one or more detents. In the example system of FIG. 10, the outer diameter of the stem 123 of the main head body 120 is less than the inner diameter of the central aperture 112 so that there is a clearance between the stem 123 and an inner wall of the central aperture 112. This clearance allows for a stationary feedback component (e.g., first detent) 150 on the inner wall of the central aperture 112 and a moveable feedback component (e.g., second detent) 151 on the stem 123. The role of the feedback components 150, 151 is to signal the user when a threshold distance has been traversed by the main head body 120 relative to the elongated portion 110. The feedback signal may be an auditory signal or a physical signal such as a click or snap that occurs as the moveable feedback component 151 is moved past the stationary feedback component 150.

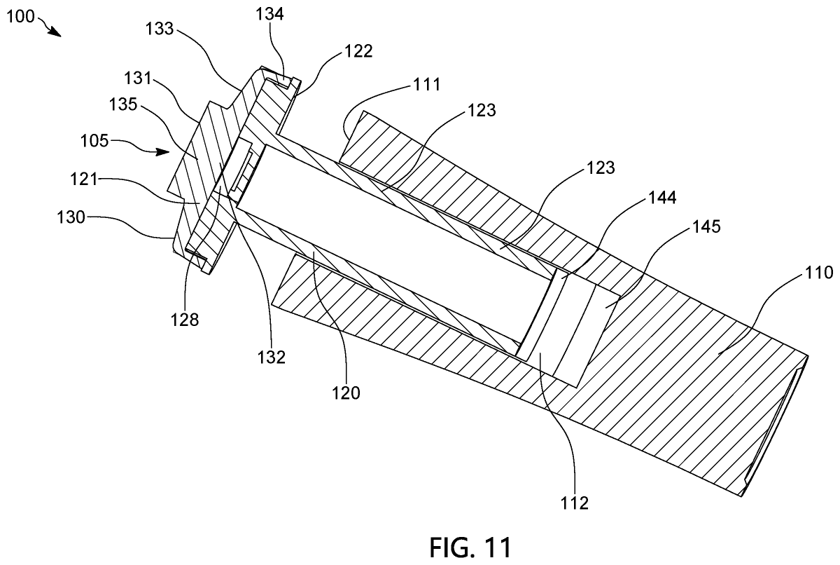

In some examples, and as shown in FIG. 11, magnets may be used as biasing and/or feedback components. More specifically, one or more magnets 144 are located on the stem 123 of the main head body 120. One or more magnets 145 may be placed within the central aperture 112, most preferably at an end of the central aperture 112 furthest away from the first surface 111 of the elongated portion 110. As the main head body 120 is moved relative to the elongated portion 110 such that the distance between the second surface 122 of the main head body 120 and the first surface 111 of the elongated portion 110 shrinks, the magnets 144, 145 may provide a resistive force as they try to repel each other. This resistive force may signal to the user that sufficient action on their part has been taken to apply the cosmetic substance to the skin below the uppermost layers of the epidermis, as well as to ensure that the array of microstructures comprising the cosmetic substance have indeed been applied as intended.

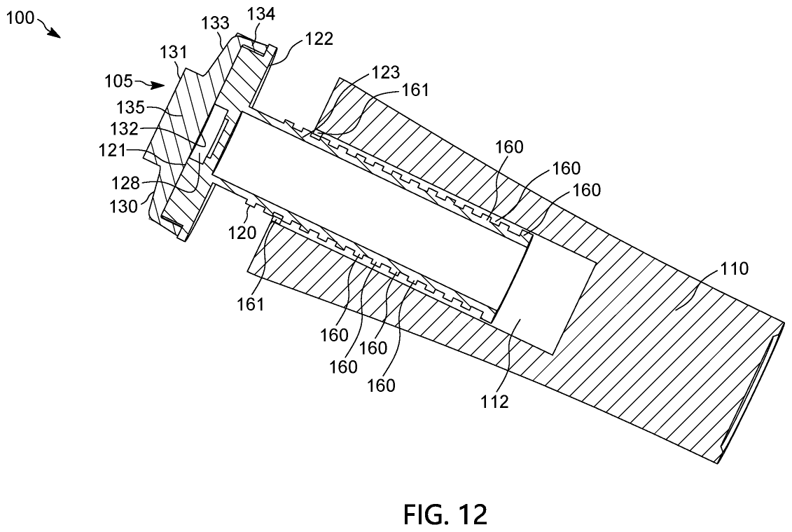

A ratcheting system may be used as both a biasing and a feedback mechanism, as shown in FIG. 12. Again, as with the example system shown in FIG. 10, there is clearance between the stem 123 and the inner wall of the central aperture 112. On the stem 123, there are many feedback features 160 in a line or array. At least one stationary feedback feature 161 is located on the inner wall of the central aperture 112, and the position of the stationary feedback feature(s) 161 is aligned with the multiple feedback features 160 on the stem 123. A tab attached to the stem 123 in a slot on the elongated portion 110 (not shown in FIG. 12) may be part of the system and used to move the main head body 120 relative to the elongated portion 110. As the main head body 120 moves relative to the elongated portion 110, the feedback features 160, 161 cause a sensation and/or create a sound that indicates to the user that force is being applied to the applicator tip 130 of the system. The feedback features 160, 161 may also provide a resistive force, so that there may be minimal slippage or relaxation in the force applied by the system on the applicator tip 130 as it is pressed against skin.

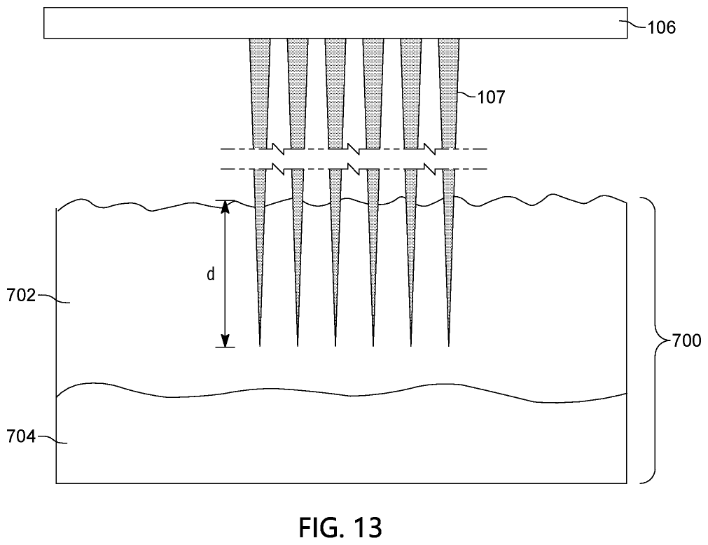

FIG. 13 is a schematic showing a substrate or chip 106 supporting an array of microneedles 107 which are inserted into skin 700 using a system 100 in accordance with various examples. The portions of the skin 700 shown are the epidermis 702 and the dermis 704. The microneedles are shown inserted to a depth d into the epidermis 702. The depth d may be between 5 μm (micrometers) and 50 μm (micrometers), such as between approximately 10 μm (micrometers) and approximately 45 μm (micrometers), such as between approximately 15 μm (micrometers) and 40 μm (micrometers), or such as between 25 μm (micrometers) and 30 μm (micrometers). The microstructures (i.e., microneedles) may range in length from about 300 μm (micrometers) to approximately 500 μm (micrometers), such as from about 325 μm (micrometers) to about 475 μm (micrometers), such as from 350 μm (micrometers) to approximately 450 μm (micrometersw including from about 375 μm (micrometers) to approximately 425 μm (micrometers). The microstructures (i.e., microneedles) may have a base ranging from 50 μm (micrometers) to approximately 100 μm (micrometers), such as from approximately 40 μm (micrometers) to approximately 90 μm (micrometers), such as from approximately 30 μm (micrometers) to approximately 80 μm (micrometers), including from approximately 40 μm (micrometers) to approximately 70 μm (micrometers). In some approaches or implementations, the microneedles may have a length of between approximately 90 μm (micrometers) and 110 μm (micrometers), such as between approximately 95 μm (micrometers) and approximately 105 μm (micrometers), such as between approximately 100 μm (micrometers) and approximately 100 μm (micrometers). The aspect ratio of the microstructures may range from 4:1 to 8:1 (height to base), such as from approximately 9:2 to approximately 15:2 (height to base), including from approximately 5:1 to 7:1 (height to base). For reference, an average human hair has a diameter on the order of approximately 80 μm (micrometers) to approximately 100 μm (micrometers). As mentioned, the microneedles, or microstructures, may be solid or hollow. If hollow, the microstructures may each have a channel of a diameter 0.001 times that of the base of the structure, that is to say on a nanometer scale. Additionally, for hollow microneedles, a reservoir for the cosmetic substance to be applied may be present on the substrate or chip 106. The microstructures, or microneedles, may be conical in shape, or they may have another shape which depends on the materials used in fabricating the microstructure arrays as well as the technique. Other shapes may include columns with sharpened or tapered ends, pyramids, truncated square pyramids, prisms with sharpened or tapered ends, and the like. The sharpened or tapered ends may have a radius of curvature on the order of 1 μm.

In use, the system may be provided in a kit with the cosmetic substance 105 to be applied. When using the kit, a user fits an elongated portion 110 with a main head body 120. An applicator tip 130 is provided with a cosmetic substance 105 on a first surface 131, with the cosmetic substance 105 being an array of microstructures or microneedles 107 of the cosmetic substance that is supported on a substrate 106. The applicator tip 130 may be preloaded with the substrate 106 and array of microstructures 107 of cosmetic substance. In such kits, multiple applicator tips may be provided. Alternatively, or additionally, one or two applicator tips 130 may be provided with a kit and an adhesive surface on the surface 131 of the applicator tip 130, the substrate 106, or both may allow for temporarily fixing the substrate 106 to the applicator tip 130 for use in the system. When ready to use the system, a user places the applicator tip 130 with cosmetic substance 105 into the recess 124 in the main head body 120. Grasping the elongated portion 110, the user presses the applicator tip 130 onto a selected area of skin, driving the each microneedle 107 of the microstructure array to a depth d into the skin, with the depth d being sufficient to infiltrate the epidermal layer of the skin, such as between about 10 μm (microns) and 50 μm (microns), including between 10 μm and 45 μm or between 30 μm and 50 μm. A predetermined threshold force applied to the applicator tip 130 may be a maximum force which may cause damage to skin tissue or the microneedles to penetrate beyond the epidermal layer. A feedback mechanism or component may emit a sound, such as a clicking sound (e.g., and audible click), or produce a sensation such as a click or resistive force indicating when the predetermined threshold force has been met or overcome. The user may cease to drive the elongated portion 110 towards the skin when experiencing the feedback and either hold the system stationary or retract the elongated portion 110. Then, the user may move the system to another area of skin and reapply the applicator tip 130 to the skin using the same array of cosmetic substance containing microstructures 107. Alternatively, the user may change the applicator tip 130 or substrate (chip) 106 before applying the applicator tip 130 to another area of skin. In some instances, a user may wish to contact the same area of skin with the applicator tip 130 multiple times, either with the same array of cosmetic substance or with a new array. In some such instances, the user may simply drive the elongated portion 110 toward and away from the skin.

It is desirable to deliver the cosmetic substance to the epidermis 702 because there are no nerves in this layer of skin and delivery into the skin in this manner allows for both the preservation of the active ingredients of the cosmetic substance until the moment of use and targeted delivery of the active ingredients. Thus, this method of substance delivery should be more effective than other means of applying the cosmetic substance without being any more painful than conventional application methods.

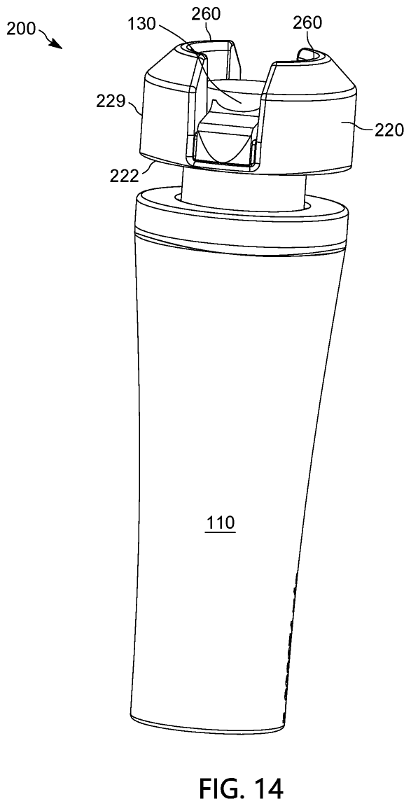

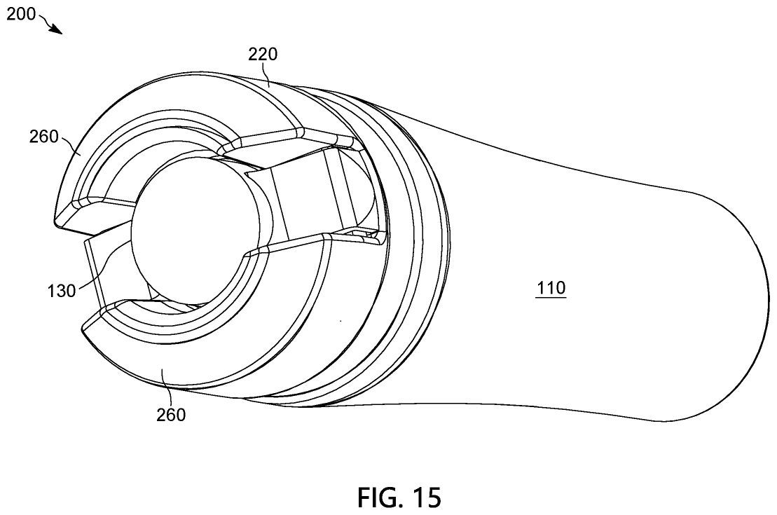

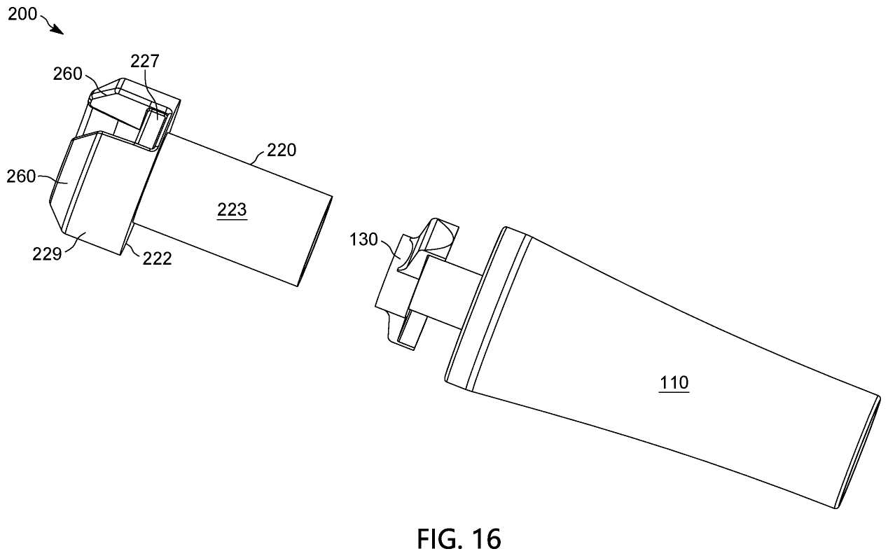

An example system 200 for applying a cosmetic substance to skin is shown in FIGS. 14-18 which includes an elongated portion 110, an applicator tip 130, and a main head body 220 with protruding portions 260 which extend from the sidewall 229 of the main head body 220. The main head body 220 has a first surface 221 which is the surface upon which the central portion of an installed applicator tip is seated when the system is in use. Additionally, the main head body 220 has a second surface 222 which abuts the handheld portion 110 when the system is in the compressed configuration.

The raised portions, the protruding portions 260 extend from the main head body 220 beyond the first surface 221 of the main head body 220. The raised portions (protruding portions) 260 taper from a first diameter at the first surface 221 to a second outer diameter at the free end, where a contacting surface 265 is located. The first diameter may be equal to the diameter of the sidewall 229 of the main head body 220. The raised portions 260 may encircle at least a portion of the applicator tip 130. In use, an example system 200 may deliver cosmetic substance through depressing the applicator tip 130 against the skin with the raised portions 260 encircling the applicator tip 130 while the cosmetic substance is delivered.

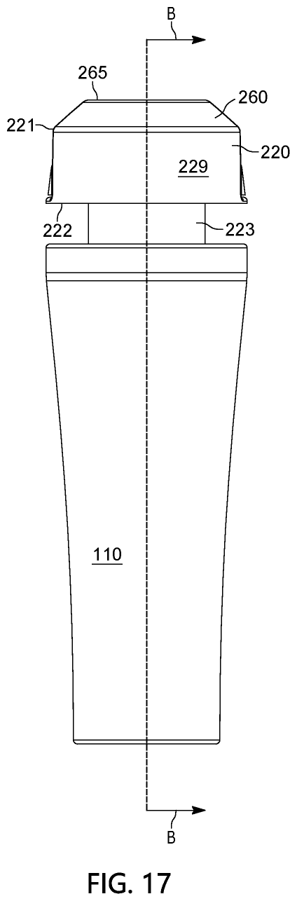

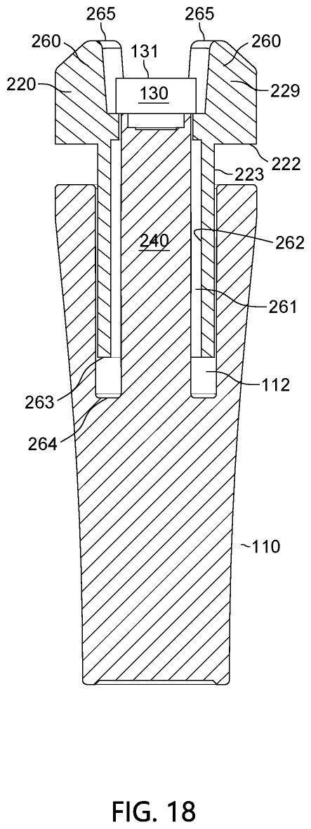

FIG. 18 is a cross-sectional view of the system 200 shown in FIG. 17 taken along the line B-B. The system 200 in FIG. 18 is shown with a clearance 261 between an inner wall 262 of the stem 223 and an inner portion 240 connected to the elongated portion 110. In the system 200 shown in FIG. 18, biasing components or mechanisms may be located in any of the following areas: the clearance volume 261, along the inner wall 262 of the stem 223, at the base of the stem 263, and a base 264 of the central aperture 112. The height which the protruding portions 260 rise above the first surface 131 of the applicator tip 130 are such that when the system 200 is fully assembled and in the compact configuration, the first surface 131 of the applicator tip 130 is surrounded by the contacting surface 265 of the protruding portions 260.

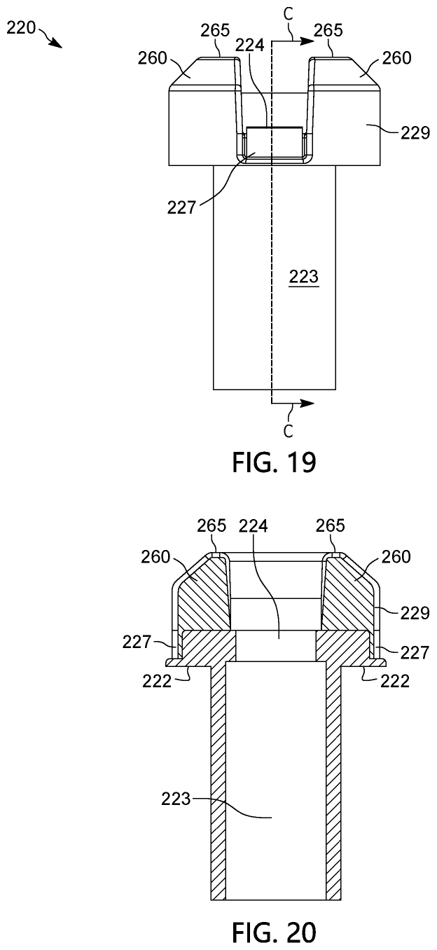

FIG. 19 and FIG. 20 show the main head body 220 in detail. FIG. 20 is a cross-sectional view of the main head body 220 of FIG. 19 taken along the line C-C. The protruding portions 260 are shaped to surround the center portion 135 of the applicator tip 130 and the center portion of the recess 224. The protruding portions 260 are shaped as a circular extension of the sidewall 229 with cut-outs for the side portions and the tab receiving portions 227 of the recess 224 on the main head body 220. The protruding portions 260 rise above the level of the first surface 131 of the applicator tip 130 when installed in the system.

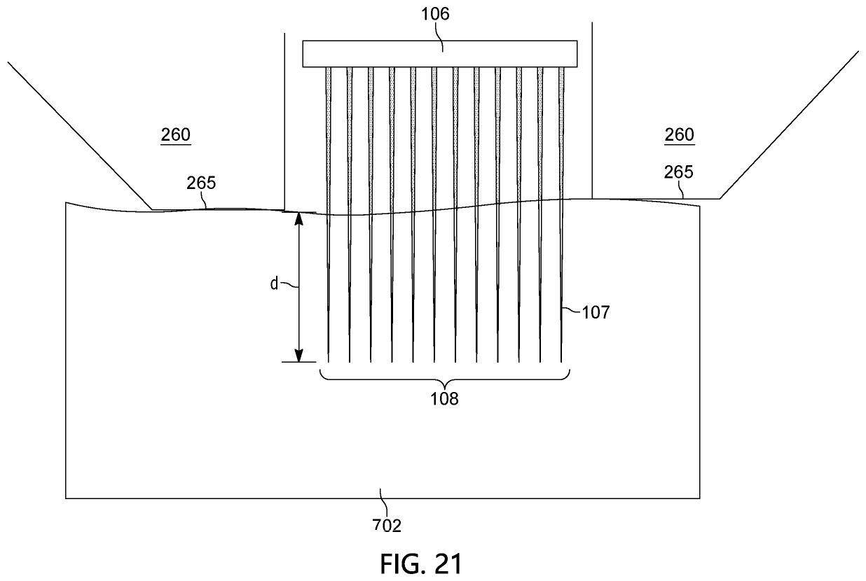

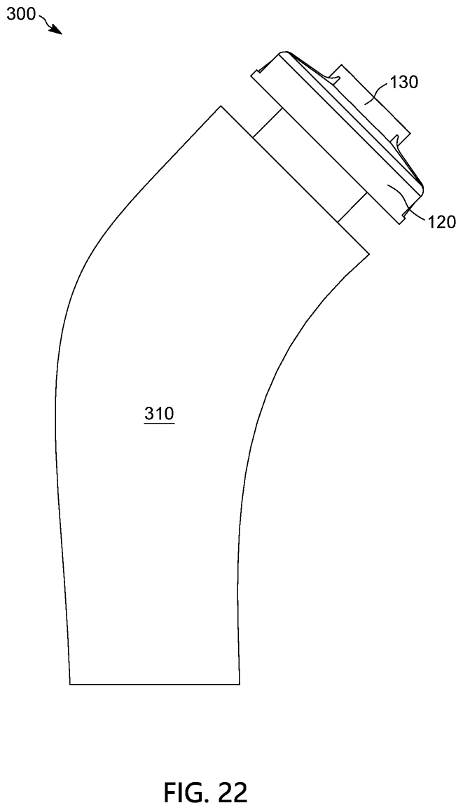

FIG. 21 shows the contacting surfaces 265 of the protruding portions 260 of the system 200, along with an array 108 of microstructures 107 comprising the cosmetic substance. The array 108 of microstructures (microneedles) 107 is supported on a substrate 106 affixed to the applicator tip. When in use, the contacting surfaces 265 of the protruding portions 260 press up against the skin to tighten the skin to which the cosmetic substance will be applied. In other words, the skin is pulled taut when force is applied during deliver of the cosmetic substance to the skin. This pre-tensioning of the skin may allow for more uniform application of the cosmetic substance to the skin as the microstructures (i.e., microneedles) are inserted into the epidermis 702 to a depth d. The pre-tensioning of the skin allows for insertion of the microstructures in a direction substantially perpendicular to the skin surface. As described with respect to FIG. 13, the depth d may range from approximately 5 μm to approximately 50 μm.

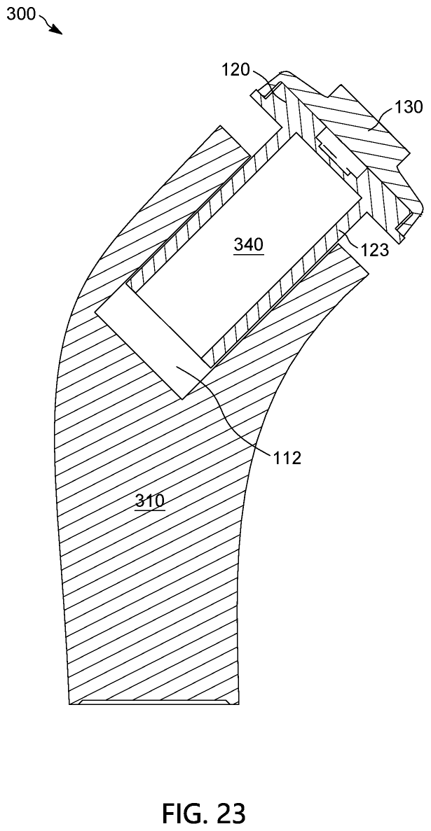

FIGS. 22 and 23 show an example system 300 for the application of cosmetic substance to skin with a portion grasped by a user (hand-held portion) 310 that is curved. The cross-sectional view of FIG. 23 shows the relative position of a main head body 120, the hand-held portion 310, and the applicator tip 130. A stem 123 of the main head body 120 fits into a central aperture 112 and a volume 340 is created when the system is fully assembled. The volume 340 may be fitted with a biasing/feedback component or mechanism as shown in FIGS. 9-12. In use, when the system 300 is assembled and the applicator tip 130 with an array of microstructures comprising the cosmetic substance is contacted with skin, the user moves the hand-held portion 310 toward the skin until feedback indicates that the predetermined maximum force has been applied. That is to say that the force is sufficient to drive the array of microstructures comprising the cosmetic substance to a predetermined depth, of no greater than 50 μm, into the epidermis. In some example systems, the microstructures comprising the cosmetic substance are microneedles of microcrystalline cosmetic material, and the cosmetic material dissolves at least partially when the array of microstructures are inserted into the skin.

As described herein, the cosmetic substance to be applied to skin may be in the form of an array of microstructures with the cosmetic substance. In some implementations, the microstructures may be microneedles, and the microneedles may have any of various configurations or constructions. The microneedles may be coated with the cosmetic substance, may each have a nanometer scale channel through which the cosmetic substance flows, and/or may be microneedles formed of solidified cosmetic substance. The solidified cosmetic substance may be formed into crystalline microneedles or amorphous, or glassy, microneedles. Alternatively, or additionally, the solidified cosmetic substance may include a frozen suspension of an effective or active ingredient. The array of microstructures may be circular, square, or any other polygon in footprint. There may be one type of microstructure in each array, as in only solid microneedles composed of microcrystalline cosmetic substance, or there may be multiple types of microstructures in each array, such as a combination of solid microneedles coated with cosmetic substance interspersed with solid microneedles composed of microcrystalline cosmetic substance. Other configurations of the arrays and mixture of types of microstructures of cosmetic substance for application to skin are also possible.

Cosmetic substances which can be used with the systems, kits, and methods described herein may include hyaluronic acid, polysaccharides, alpha hydroxy acid, glycolic acid, lactic acid, mandelic acid, citric acid, fatty acids, ceramides, waxy lipid molecules, peptides, and the like. The cosmetic substances may be applied to an epidermis layer of the skin using coated microstructures (microneedles), where the coating includes a gel or dried layer of the cosmetic substance. Alternatively, or additionally, the cosmetic substances may be applied using hollow microstructures or microneedles, laid out in an array on a substrate or chip and where the cosmetic substance flows through the microneedles for delivery to an epidermal layer of the skin. In some implementations, the cosmetic substance may be solidified (e.g., frozen or crystallized) and formed into microstructures supported on a substrate or chip.

The various systems described herein may be provided as a kit including a handheld device and a chip with a cosmetic substance to be applied to skin. The handheld device may include an elongated portion, such as elongated portions 110 and 310 shown in FIGS. 4, 22, and 23. The handheld device may also include a main head body such as those shown in FIGS. 5 and 19. Additionally, the handheld device may include an applicator tip, such as those shown in FIGS. 6A, 6B, 7A, and 7B. The kit may include one or more chips, or substrates, which support cosmetic substance to be applied to skin. The cosmetic substance may be deposited on the substrate as an array of microstructures, such as microneedles, such as described hereinabove.

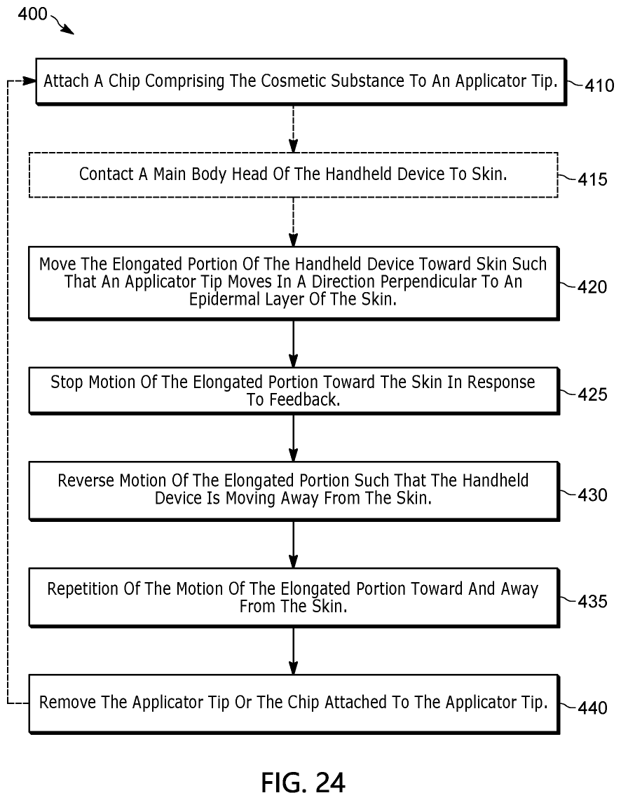

FIG. 24 illustrates a method 400 for using the systems, devices, and kits described herein to deliver a cosmetic substance to skin according to various examples. Systems for use with the method can include a handheld device with an elongated portion and a main head portion, as well as an applicator tip. Such systems can be part of kits which also include one or more chips or substrates with the cosmetic substance to be applied to skin. In the method, a chip with the cosmetic substance is attached to an applicator tip of a system for applying a cosmetic substance, as in a step 410. In some implementations, a main body head of the handheld device is contacted to skin, as in a step 415. Next, the elongated portion of the handheld device is moved toward the skin such that the applicator tip moves in a direction perpendicular to an epidermal layer of the skin, as in a step 420. This motion of the applicator tip substantially perpendicular to the skin may be desirable to optimize delivery of the cosmetic substance to a target portion of the skin. A user moves the elongated portion toward the skin until receiving feedback (e.g., haptic feedback, sensation, sound), at which point, the user stops motion of the elongated portion, as in a step 425. Next, the user reversed motion of the elongated portion, moving the handheld device away from the skin, as in a step 430. There can be a pause in motion of the handheld device between stopping at the motion of the handheld device and reversing motion. This pause in motion can be timed to allow for delivery of the cosmetic substance, such that a user pauses for a predetermined number of seconds. Repetition of the motion of the elongated portion toward and away from the skin can be performed multiple times, as in a step 435. This can be done with variable timing between applications or contact with the skin and the cosmetic substance mounted on the applicator tip. Alternatively, or additionally, the method may include removing the applicator tip or the chip attached to the applicator tip, as in a step 440, and further attaching another chip to the applicator tip. In various methods, the chip or substrate may dissolve, and the method may include attaching a new chip to the application without removal of a used or spent chip.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . aa”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A handheld device for applying a cosmetic substance to skin, the handheld device comprising:
an elongated portion capable of being grasped by a user;
a main head body comprising a biasing mechanism and being operably coupled with the elongated portion, the main head body further comprising a top side; and
an applicator tip capable of being seated on the top side of the main head body and further capable of supporting the cosmetic substance to be applied to the skin.

2. The handheld device of claim 1, wherein the biasing mechanism of the main head body is operable to restrict infiltration of the cosmetic substance into an epidermal layer of the skin until a predetermined threshold force is applied to the applicator tip.

3. The handheld device of claim 2, wherein the handheld device is capable of delivering the cosmetic substance to a depth of between approximately 10 μm and 45 μm into the epidermal layer.

4. The handheld device of claim 1, wherein the main head body has a recess for receiving the applicator tip and a stem which fits into a central aperture of the elongated portion.

5. The handheld device of claim 1, further comprising a feedback indicator operable to provide an indication when a predetermined maximum force is applied to the applicator tip.

6. The handheld device of claim 5, wherein the indication of feedback comprises at least one of a sound, a clicking sensation, an audible click, or a resistive force.

7. The handheld device of claim 6, wherein the resistive force is caused by a detent in the handheld device between the main head body and the elongated portion.

8. The handheld device of claim 5, wherein the predetermined maximum force is a force beyond which damage to the skin and tissues beneath the skin may occur.

9. The handheld device of claim 1, wherein the cosmetic substance has a microcrystalline structure.

10. The handheld device of claim 1, wherein the handheld device is capable of delivering the cosmetic substance through depressing the applicator tip against the skin with a first pressure greater than a threshold minimum pressure until a second pressure less than or equal to a predetermined maximum force is applied to the skin, wherein the handheld device is capable of being held by a user applying pressure.

11. The handheld device of claim 10, further comprising a raised portion of the main head body, the raised portion encircling at least a portion of the applicator tip.

12. The handheld device of claim 11, wherein the raised portion of the main head body has a free end which extends from the main head body beyond the applicator tip when no force is applied, the main head body tapering from a first outer diameter to a second outer diameter at the free end, and further wherein the raised portion of the main head body is capable of pulling the skin taut when force is applied to the skin during delivery of the cosmetic substance.

13. A method of delivering a cosmetic substance to skin, the method comprising:
attaching a chip comprising the cosmetic substance to an applicator tip of a handheld device, the handheld device comprising:
an elongated portion capable of being grasped by a user;
a main head body comprising a biasing mechanism and being operably coupled with the elongated portion, the main head body further comprising a top side; and the applicator tip configured to be seated on the top side of the main head body and further capable of supporting the cosmetic substance to be applied to the skin;
contacting the main head body of the handheld device to the skin;
moving the elongated portion toward the skin such that applicator tip moves in a direction perpendicular to an epidermal layer of the skin;
stopping motion of the elongated portion toward the skin in response to feedback;
reversing motion of the elongated portion such that the handheld device is moving away from the skin; and
repetition of the motion of the elongated portion toward the skin and away from the skin.

14. The method of claim 13, further comprising delivery of the cosmetic substance in a single location of the skin.

15. The method of claim 13, wherein the repetition of the motion of the elongated portion is determined by a user of the handheld device.

16. A kit for applying a cosmetic substance to skin, the kit comprising:
a handheld device for applying the cosmetic substance comprising:
an elongated portion capable of being grasped by a user;
a main head body comprising a biasing mechanism and being operably coupled with the elongated portion, the main head body further comprising a top side; and
an applicator tip capable of being seated on the top side of the main head body and further capable of supporting the cosmetic substance to be applied to the skin;
and
a chip comprising the cosmetic substance to be applied to the skin.

17. The kit of claim 16, wherein the chip comprises a microneedle composed of the cosmetic substance.

18. The kit of claim 17, wherein the handheld device is capable of inserting the microneedle into an epidermal layer of the skin when a pressure at or above a predetermined threshold minimum pressure is exerted on the applicator tip of the handheld device.

19. The kit of claim 18, wherein the handheld device is capable of inserting the microneedle to a depth of between approximately 30 μm and 50 μm into the epidermal layer.

20. The kit of claim 16, wherein the chip is part of an array of chips comprising the cosmetic substance.
